(19) United States
(12) Patent Application Publication
(10) Pub. No.: US 2024/0277886 A1
(43) Pub. Date: Aug. 22, 2024

(54) AIR CONDITIONING EQUIPMENT

(71) Applicant: NIPPON TELEGRAPH AND TELEPHONE CORPORATION, Tokyo (JP)

(72) Inventors: Asuka MIYAKE, Musashino-shi, Tokyo (JP); Hitoshi SESHIMO, Musashino-shi, Tokyo (JP); Ryuji YAMAMOTO, Musashino-shi, Tokyo (JP); Tomohiro TANIGUCHI, Musashino-shi, Tokyo (JP); Ayako IWAKI, Musashino-shi, Tokyo (JP); Satoshi NARIKAWA, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP); Nobutomo HANZAWA, Musashino-shi, Tokyo (JP)

(73) Assignee: NIPPON TELEGRAPH AND TELEPHONE CORPORATION, Tokyo (JP)

(21) Appl. No.: 18/693,133

(22) PCT Filed: Sep. 30, 2021
(86) PCT No.: PCT/JP2021/036048

§ 371 Date: Mar. 18, 2024

(51) Int. Cl. A61L 9/20; F24F 8/22

(52) U.S. Cl. CPC A61L 9/20; F24F 8/22; A61L 2209/12; A61L 2209/16

(57) ABSTRACT

An object of the present invention is to provide an air conditioner unit that allows for greater flexibility in designing the shape of the unit and is capable of reducing the cost of lamp replacement.

An air conditioner unit according to the present invention includes an optical fiber group 17 formed by bundling a plurality of optical fibers 16 emitting a part of propagating light from side faces thereof; an air duct 10 that incorporates the optical fiber group 17 in a space 13 through which air passes; and a light source 30 that supplies ultraviolet light to each of the optical fibers 16. Since the intensity of the light is inversely proportional to the square of the distance from the light source, if the same sterilization effect is to be obtained, the irradiation intensity of the ultraviolet light can be reduced when the irradiation distance is shorter. In other words, in this air conditioner unit, since the plurality of optical fibers 16 are arranged in the air duct 10, the air (air flow) in the air duct 10 can be sterilized by the ultraviolet light of the LED having a small output from the light source 30.

### TECHNICAL FIELD

The present disclosure relates to an air conditioner unit for decontaminating air in an air duct.

### BACKGROUND ART

For the purpose of preventing infectious diseases, there is an increasing demand for systems for sterilization and inactivation of viruses using ultraviolet light. In the present embodiment, “decontamination” is assumed to include sterilization and virus inactivation.

An air circulation-type ultraviolet air sterilizer is disclosed in NPL 1. The sterilizer sucks up air from below and performs air sterilization by irradiating a rod-shaped UV lamp in a cylindrical interior while the air is sucked up from below and discharged from above.

### CITATION LIST

### Non Patent Literature

[NPL 1] Wet site, Iwasaki Electric Co., Ltd., Air circulation-type ultraviolet purifier, https://www.iwasaki.co.jp/optics/sterilization/air/air03.html (searched on Apr. 6, 2021)

### SUMMARY OF INVENTION

### Technical Problem

In order to perform air sterilization, it is generally necessary to use a high-output UV lamp. However, the UV lamp is rod-shaped and its length is determined by the standard. Therefore, the ultraviolet air sterilizer is shaped to fit the size of the UV lamp, limiting design flexibility. Moreover, the life of the UV lamp is approximately one year. Therefore, the ultraviolet air sterilizer has a problem that the cost for lamp replacement is high.

Therefore, in order to solve the foregoing problem, an object of the present invention is to provide an air conditioner unit that allows for greater flexibility in designing the shape of the unit and is capable of reducing the cost of lamp replacement.

### Solution to Problem

In order to achieve the above-mentioned object, the air conditioner unit according to the present invention has a bundle of optical fibers arranged in a duct and is configured to supply ultraviolet light to the optical fibers from the outside.

Specifically, the air conditioner unit according to the present invention includes an optical fiber group formed by bundling a plurality of optical fibers emitting supplied ultraviolet light, an air duct incorporating the optical fiber group in a space through which air passes, and a light source supplying the ultraviolet light to each of the optical fibers. The optical fibers may be a side irradiation type, an end irradiation type, or a type with both functions.

The light source of the air conditioner unit is characterized by being a light emitting diode (LED).

The diameter of a conventional UV lamp is several centimeters. The diameter of the optical fibers is approximately several hundred μm, which is approximately 1/100 of the diameter of the UV lamp. Therefore, an air duct in which a single UV lamp is placed can accommodate a plurality of optical fibers at high density. Furthermore, the optical fibers are small in diameter and can be bent, which allows for greater flexibility in the size and shape of the unit.

Here, the cross section of the air duct is considered as shown in FIG. 1. FIG. 1(A) shows an example in which one UV lamp 15 is disposed in an air duct 10. Reference numeral 13 denotes an air flow path. In this case, a place away from the UV lamp 15 (for example, the vicinity of a duct wall) is a place where the irradiation distance of the ultraviolet light is long. FIG. 1(B) shows an example in which a plurality of optical fibers 16 are installed in the air duct 10. In this case, since the optical fibers 16 are dispersedly arranged in the air duct 10 (arranged so that a gap through which air passes is present between the optical fibers), the irradiation distance of the ultraviolet light is shortened in any place.

Since the intensity of the light is inversely proportional to the square of the distance from the light source, if the same sterilization effect is to be obtained, the irradiation intensity of the ultraviolet light can be reduced when the irradiation distance is shorter. In other words, the optical fibers can be sterilized by the LED having a small output. For example, even a UV-LED with a current maximum output of approximately 0.1 w is theoretically sufficient to achieve a sterilizing effect.

Accordingly, the present invention can provide an air conditioner unit that allows for greater flexibility in designing the shape of the unit and is capable of reducing the cost of lamp replacement.

If the optical fibers are of the side irradiation type, the air in the air duct is sterilized by ultraviolet light only for a period of time when the air passes through the optical fiber group. On the other hand, if the optical fibers are of the end irradiation type, when the lengths of all the optical fibers are equal and the tips (end portions) of the optical fibers are at the same position in the longitudinal direction of the air duct, the irradiation region of the ultraviolet light becomes one place in the longitudinal direction of the air duct, and the air in the air duct is sterilized by the ultraviolet light only at the moment when the air passes through the end portions of the optical fibers. For this reason, sterilization may be insufficient depending on the intensity of the ultraviolet light and the flow velocity of the air.

Therefore, the lengths of the respective optical fibers are made different, and the end portions of the optical fibers are located at different positions for the respective optical fibers, in the longitudinal direction of the air duct. Since the end portions of the respective optical fibers are located at different positions in the longitudinal direction of the air duct, the irradiation region of the ultraviolet light has a width in the longitudinal direction of the air duct, and the air in the air duct is sterilized by the ultraviolet light only for a period of time when the air passes through the region. That is, the same effect as that of the side irradiation-type optical fibers can be obtained. In addition, since the intensity of the ultraviolet light from end faces of the optical fibers is stronger than the intensity of the ultraviolet light from side faces, bringing about the advantage of having a higher sterilization effect.

When the optical fibers are of the side irradiation type, the end portions of the optical fibers may be directed in arbitrary directions respectively.

The above inventions can be combined as much as possible.

### Advantageous Effects of Invention

The present invention can provide an air conditioner unit that allows for greater flexibility in designing the shape of the unit and is capable of reducing the cost of lamp replacement.

### BRIEF DESCRIPTION OF DRAWINGS

FIG. 1 is a diagram for explaining a cross section of an air duct.

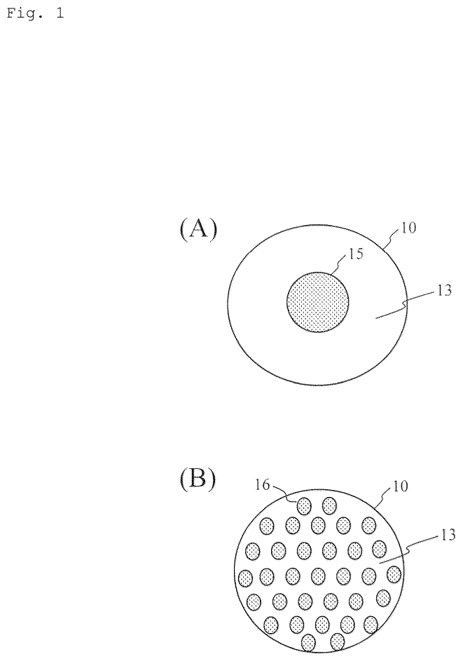

FIG. 2 is a diagram for explaining an air conditioner unit according to the present invention.

FIG. 3 is a diagram for explaining the air conditioner unit according to the present invention.

FIG. 4 is a diagram for explaining the air conditioner unit according to the present invention.

### DESCRIPTION OF EMBODIMENTS

Embodiments of the present invention will be described with reference to the accompanying drawings. The embodiments described below are examples of the present invention, and the present invention is not limited to the following embodiments. Note that, in the present specification and the drawings, components having the same reference numerals indicate the same components.

### Embodiment 1

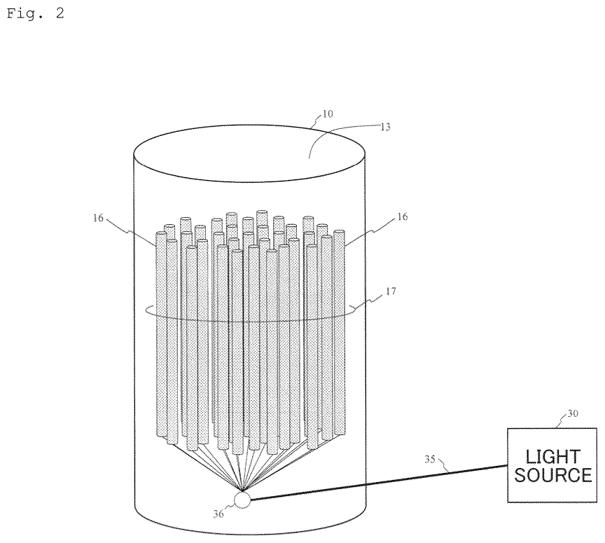

FIG. 2 is a diagram for explaining an air conditioner unit of the present embodiment. The air conditioner unit includes:
an optical fiber group 17 formed by bundling a plurality of optical fibers 16 emitting a part of propagating light from side faces thereof;
an air duct 10 that incorporates the optical fiber group 17 in a space 13 through which air passes; and
a light source 30 that supplies ultraviolet light to each of the optical fibers 16.

The air conditioner unit is, for example, an air circulation-type air purifier. The air purifier has an air pump, not shown. The air pump pushes air taken in from the room into an air flow path 13 in the air duct 10.

The optical fiber group 17 formed by bundling the plurality of optical fibers 16 is arranged in the air flow path 13. The optical fibers 16 of the optical fiber group 17 are not brought into close contact with adjacent optical fibers, so a gap through which air passes is provided. The optical fibers 16 are optical fibers that allow propagating light to leak from side faces thereof. For example, the optical fibers 16 contain a scatterer in the cores thereof, light propagating through the cores is scattered by the scatterers, and the scattered light is emitted from the side faces through the cladding.

The light source 30 is an LED for outputting ultraviolet light. The ultraviolet light that is output from the light source 30 is branched by an optical branching device 36 through a transmission line 35 and supplied to each optical fiber 16.

As described above, since the intensity of the light is inversely proportional to the square of the distance from the light source, if the same sterilization effect is to be obtained, the shorter the irradiation distance, the smaller the irradiation intensity of the ultraviolet light can be. That is, since the plurality of optical fibers 16 are arranged in the air duct 10 as shown in FIG. 2, the air (air flow) in the air duct 10 can be sterilized by the ultraviolet light of the LED having a small output from the light source 30.

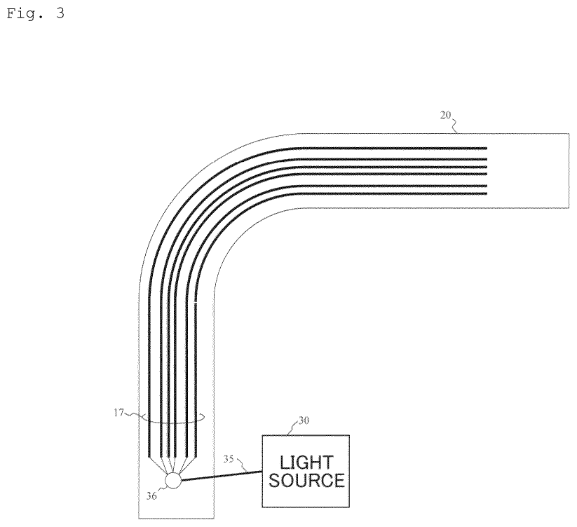

Further, since the optical fibers 16 have a small diameter and can be bent, the optical fiber group 17 can be installed over a long section of several meters or more including a curved section, in a piping duct 20 of the air conditioner unit, as shown in FIG. 3. That is, the ultraviolet light can be radiated to the air (air flow) over a long section. Therefore, the irradiation time can be prolonged, and the integrated irradiation amount can be increased even if the output of the ultraviolet light of the light source 30 is small.

### Embodiment 2

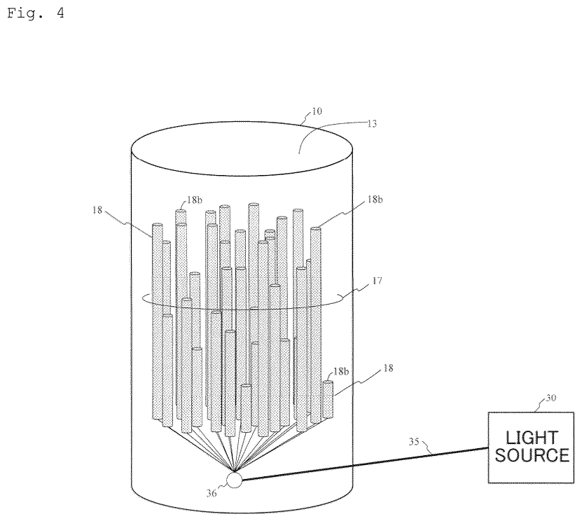

Embodiment 1 has described the case where the optical fibers 16 are of the side irradiation type. The present embodiment will described end irradiation-type optical fibers 18 with reference to FIG. 4.

In the case of the end irradiation-type optical fibers 18, if the lengths of all the optical fibers are the same, the end portions of the optical fibers become aligned in the longitudinal direction of the air duct 10, resulting in only one UV irradiation area. In such a state, depending on the intensity of the ultraviolet light and air velocity, the integrated amount of ultraviolet light irradiated is small, resulting in insufficient sterilization.

Therefore, in the air conditioner unit of the present embodiment using the end irradiation-type optical fibers 18, the lengths of the respective optical fibers 18 are made different, and the positions of end portions 18b of the respective optical fibers 18 are made different in the longitudinal direction of the air duct 10. Thus, the ultraviolet light irradiation region can be expanded in the longitudinal direction of the air duct 10 by dispersing the positions of the end portions 18b, and the air in the air duct is sterilized by the ultraviolet light only for a period of time when the air passes through the region.

Here, the end portions 18b of the optical fibers 18 may be processed into a shape (for example, uneven processing) for scattering the ultraviolet light in a plurality of directions.

The end portions 18b of the optical fibers 18 may be bent perpendicularly to the longitudinal direction of the air duct 10 (the bending direction may be arbitrary), and may be a shaped portion that allows the ultraviolet light to be radiated perpendicular to the air flow.

### Effects

The air conditioner unit according to the present invention has the following effects.
By installing a plurality of optical fibers in an air duct, a sufficient decontamination effect can be obtained even by an LED light source.
An LED light source uses less power than a UV lamp and can reduce running costs.
Furthermore, since an LED light source has a longer life than a UV lamp, the frequency of replacement can be reduced and the running costs can be reduced.
Moreover, by using an optical fiber having a long service life, the cost required for replacement can be reduced.
Since the optical fiber has a small diameter and can be bent, the degree of flexibility in designing and arranging the unit can be increased.

### REFERENCE SIGNS LIST

10 Air duct
13 Air flow path
15 UV lamp
16 Optical fiber
17 Optical fiber group
20 Piping duct
30 Light source
35 Optical transmission line
36 Optical branching device

1. An air conditioner unit, comprising:
an optical fiber group formed by bundling a plurality of optical fibers emitting supplied ultraviolet light;
an air duct incorporating the optical fiber group in a space through which air passes; and
a light source supplying the ultraviolet light to each of the optical fibers.

2. The air conditioner unit according to claim 1, wherein the light source is a light emitting diode (LED).

3. The air conditioner unit according to claim 1, wherein end portions of the optical fibers are located at different positions for the respective optical fibers, in a longitudinal direction of the air duct.

4. The air conditioner unit according to claim 3, wherein the end portions of the optical fibers are directed in arbitrary directions.
